(19) United States
(12) Patent Application Publication
(10) Pub. No.: US 2009/0316925 A1
(43) Pub. Date: Dec. 24, 2009

(54) ELECTRONIC STETHOSCOPE SYSTEM

(76) Inventors: Leonard Eisenfeld, West Hartford, CT (US); Jonathan M. Hill, West Hartford, CT (US); Ronald S. Adrezin, West Hartford, CT (US)

Correspondence Address: MICHAUD-DUFFY GROUP LLP, 306 INDUSTRIAL PARK ROAD, SUITE 206, MIDDLETOWN, CT, 06457, US

(21) Appl. No.: 12/488,828

(22) Filed: Jun. 22, 2009

Related U.S. Application Data
(60) Provisional application No. 61/074,327, filed on Jun. 20, 2008.

(51) Int. Cl. A61B 7/04

(52) U.S. Cl. 381/67

(57) ABSTRACT

An electronic stethoscope head includes a head member having a contact surface for contact with a patient's body, a transducer in the head member, and an adhesive on the contact surface. A processing system for an electronic stethoscope includes a conditioning circuit configured to receive a transducer signal from a transducer and to be capable of amplifying and/or filtering the transducer signal, to yield a conditioned signal. There is also a signal processor system configured to subject the conditioned signal to an audio editing process. Bodily sounds are detected by applying an electronic stethoscope head a patient's body; generating a patient sonograph of the patient's bodily sounds; and comparing the patient sonograph to a reference sonograph. An electronic stethoscope system may include an accessory device and control circuitry to control the accessory device when abnormal bowel sounds are detected or no bowel sounds are detected for a predetermined interval.

### CROSS-REFERENCE TO RELATED APPLICATION

This application claims the benefit under 35 U.S.C. §119(e) of U.S. Provisional Patent Application Ser. No. 61/074,327, filed Jun. 20, 2008, the disclosure of which is incorporated herein by reference.

### FIELD OF THE INVENTION

This invention relates generally to medical sensing equipment and, in particular, to equipment for monitoring bowel sounds in patients such as infants.

### BACKGROUND

A Neonatal Intensive Care Unit (NICU) provides care for infants in need of constant supervision. Most of these infants are premature, so they typically have difficulties that other infants do not encounter such as, for example, difficulties with breathing and/or digestion. Therefore, the vital signs of such infants are continuously monitored. Typically, there are four vital signs of infants that are monitored: respiratory rate, heartbeat, temperature, and oxygen saturation in blood.

Currently, there are no bowel sound recorders or analyzers in NICUs. As such, diagnosing digestive problems is difficult even for skilled medical practitioners. In an effort to monitor bowel sounds, doctors and nurses employ infant stethoscopes. The regularity of bowel sounds depends on each infant and the state or condition that they are in, for example, before or after eating, awake or asleep. The absence of bowel sounds can be an indication that an infant is unable to digest food, and thus cannot be fed in a conventional manner. Accordingly, feeding infants that present compromised digestive systems in the same manner as infants without such compromised digestive systems could be extremely hazardous to the infant's health. Further complicating diagnosis is the fact that due to the small size of an infant, caution must be exercised when placing any weight on the infant, even the end of a conventional stethoscope. Moreover, due to the tiny stature of an infant and the immaturity of their internal organs, bowel sounds may be very faint. As such, ambient noise as well as the movement of the infant may mimic bowel sounds.

Premature infants receive nutrition much earlier in the development cycle than infants with full-term delivery. Since a premature digestive system may not be receptive to nutrition, caretakers must decide whether it is safe to feed the premature infant. Bowel sounds have a different regularity for each infant before and after eating. Bowel sounds are also affected by the fullness of the stomach. The inventors have discovered that infants with normal bowel sounds have lower pitch, lower frequency sounds. Infants with troubled bowel sounds exhibit higher pitched, higher frequency irregular sounds. Gastrointestinal dysfunction has a number of causes that include immaturity of the digestive tract, birth defects, mild intolerance or allergic reaction to food, enzyme abnormality, electrolyte imbalance, abnormal vascular supply, infection, and systemic illness. Symptomatic of such dysfunction is an abnormality or lack of peristalsis, which is the pattern of smooth muscle contractions that moves materials through digestion.

Peristalsis may be monitored acoustically to determine the health of the patient's digestive tract. It is currently common practice for nurses to use traditional stethoscopes to periodically listen for and analyze bowel sounds. Unfortunately, the ability to identify the aural cues in bowel sounds is a learned skill that takes time to develop. In addition the interpretation of bowel sounds is currently entirely subjective and based on training and experience. Further, the human ear is limited in its sensitivity and specificity in detecting bowel sounds. Also, given the sometimes transient nature, some bowel sounds may not be heard. Based on these reasons, clinical acumen is limited and diagnostic findings may or may not be correct.

In general, there are two types of stethoscope heads for collecting bodily sounds, those having a head member configured as a bell and those having a diaphragm mounted on a diaphragm head. The bell transmits lower frequency sounds than the diaphragm. Many stethoscopes have a dual head design which provides both a bell and a diaphragm, such as the dual head stethoscope seen in U.S. Pat. No. 4,475,619 to Packard, dated Oct. 9, 1984, which is incorporated herein by reference as background information. An electronic stethoscope head includes a transducer that generates electronic transducer signals in response to detected bodily sounds. The electronic transducer signals may be converted into sound which is conveyed to the user via a transmission tube or otherwise. An electronic stethoscope head may allow a user to amplify lower volume signals by pressing the stethoscope more firmly against the body. In conventional systems, more pressure translates to more volume. However, due to the delicacy of infants, it is very difficult to amplify the volume of bowel sounds via the application of greater pressure.

### SUMMARY OF THE INVENTION

The present invention resides in one aspect in an electronic stethoscope head which includes a head member having a contact surface for contact with a patient's body, a transducer in the head member, and an adhesive on the contact surface.

The invention resides in another aspect in a processing system for an electronic stethoscope. The processing system includes an analog processing circuit configured to receive a transducer signal from a transducer and to be capable of processing the transducer signal such as by amplifying and/or filtering the transducer signal, to yield a conditioned signal. The processing system includes a signal acquisition system configured to digitize the conditioned signal, to filter the digitized conditioned signal and to present the digitized conditioned system to aid diagnosis of a condition of a patient.

The invention resides in still another aspect in an electronic stethoscope system which includes an electronic stethoscope head comprises a head member having a contact surface for contact with a patient's body, and a transducer and a processing system as described herein.

In another aspect, the invention relates to a method for detecting bodily sounds of a patient. The method includes applying the stethoscope head of an electronic stethoscope system as described herein to the body of the patient. A digitized conditioned signal is generated from the stethoscope head, and an analog signal is reconstructed from the digitized conditioned signal.

In yet another aspect, a method for detecting bodily sounds includes applying the stethoscope head to the body of a patient; generating a digitized conditioned signal from the stethoscope head, and applying frequency shifting and/or frequency stretching to the digitized conditioned signal to aid diagnosis.

### BRIEF DESCRIPTION OF THE DRAWINGS

FIG. 1A is a schematic cross-section of a generalized stethoscope head according to one embodiment.

FIG. 1B is a schematic cross-section of a bell-type stethoscope head according to another embodiment.

FIG. 1C is a schematic cross-section of a diaphragm-type stethoscope head according to another embodiment.

FIG. 1D is a schematic cross-section of a stethoscope head according to another embodiment.

FIG. 1E is a schematic perspective view of a stethoscope head with a removable transducer according to another embodiment.

FIG. 1F is a schematic perspective view of a paddle-style stethoscope head according to another embodiment.

FIG. 1G is a schematic cross-section of a stethoscope head which includes a microphone for noise-cancellation according to another embodiment.

FIG. 1H is a schematic representation of an electronic stethoscope system as described herein, according to one embodiment.

FIG. 1I is a schematic diagram of a stethoscope system according to one embodiment.

FIG. 1J is a schematic diagram of a stethoscope system according to another embodiment.

FIG. 2 is a schematic diagram of an electronic stethoscope system according to another embodiment of the invention.

FIG. 3 is a flowchart of a filtering algorithm for use in one embodiment.

FIG. 4 is a sonograph of an unfiltered audio file of bowel sounds of an infant before eating.

FIG. 5 is a plot spectrum sonograph of an audio file of bowel sounds of an adult (before eating), prior to filtering.

FIG. 6 is a plot spectrum sonograph of an audio file of bowel sounds of an adult (before eating), after filtering.

FIGS. 7A and 7B illustrate features of a digital Butterworth filter simulated to visualize bowel sound signals for use in another embodiment of the invention.

FIG. 8 is a plot spectrum sonograph of audio files of bowel sounds of an adult and an infant, before and after eating.

FIG. 9 is a schematic diagram of an electronic stethoscope system according to another embodiment of the invention.

FIG. 10 is a schematic diagram of a portion of the electronic stethoscope system of FIG. 9.

FIG. 11 is an oscilloscope trace of an analog signal produced by an electronic stethoscope system of FIG. 10.

FIG. 12 is a trace of a digitized version of the signal shown in FIG. 11.

FIG. 13 is a sonograph of an audio file of bowel sounds captured from an electronic stethoscope system according to the invention.

FIG. 14 is a top-level schematic of an electronic processing system for an electronic stethoscope.

FIG. 15 is a schematic of an example amplifier circuit for use in a system as shown in FIG. 14.

FIG. 16A and FIG. 16B are schematic views of digital controlled potentiometers for use in an electronic processing system.

FIG. 16C is a schematic of an analog to digital converter for use in an electronic processing system.

FIG. 17A and FIG. 17B are sonographs of an infant's bowel sounds, before and after eating, respectively.

FIG. 18A is a sonograph of normal infant bowel sounds.

FIG. 18B is a sonograph of the bowel sounds of an infant with necrotizing enterocolitis.

FIG. 18C is a sonograph of the bowel sounds of an infant with gastroesophageal reflux disease (GERD).

### DETAILED DESCRIPTION OF PREFERRED EMBODIMENTS

The present invention provides, in various aspects and embodiments, an electronic stethoscope head and an electronic processing system operatively coupled to the electronic stethoscope head for processing signals from the electronic stethoscope head, each of which find utility individually and when used together as an electronic stethoscope system, for the electronic detection, monitoring and analysis of body sounds of a patient, for example, abdominal or so-called bowel sounds, cardiac sounds, vascular sounds and respiratory sounds in adults and infants, including premature infants. In particular, the electronic stethoscope head, the electronic processing system and the electronic stethoscope system described herein are useful for detecting bowel sounds and for diagnosing bowel dysfunctions in premature infants such as functional or anatomic bowel obstruction, which may be reflected as an abnormality or lack of peristalsis. In some aspects, the invention provides a device which will detect, record, and filter bodily sounds.

In some aspects, the invention provides an electronic processing system that detects, records, analyzes and presents data that characterizes the bowel sounds, optionally continuously, in a form meaningful to medical personnel, and may help the personnel diagnose and/or prevent life threatening problems such as necrotizing enterocolitis, which is an inflammatory disease of a premature infant. In addition, early detection and diagnosis with the systems and methods described herein may allow the medical personnel to implement protective measures that eliminate or substantially minimize vomiting, gastroesophageal reflux, and pulmonary aspiration of gastric contents.

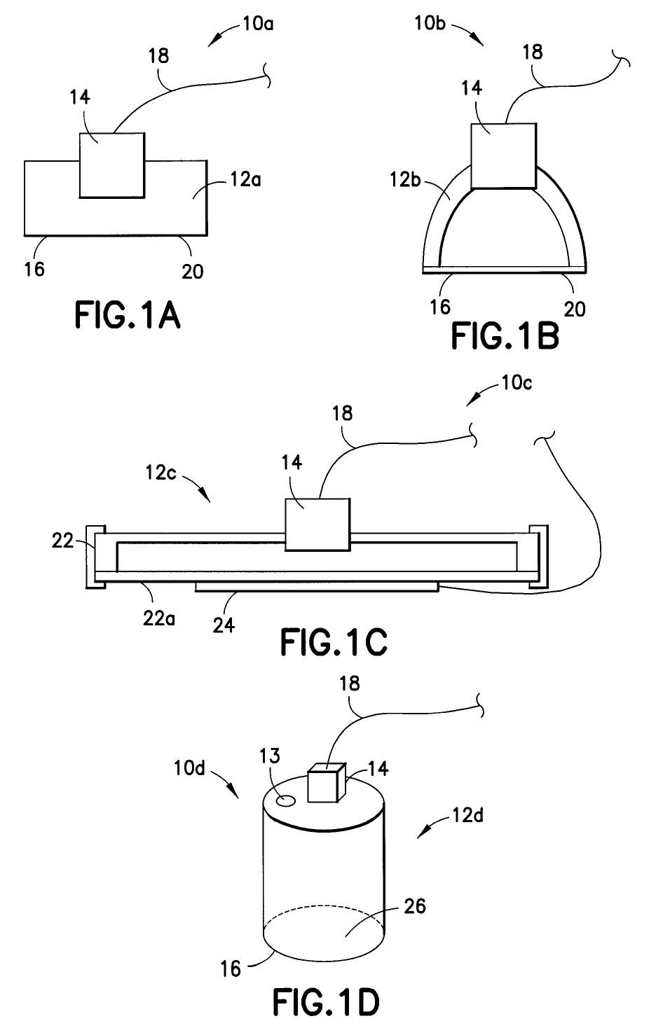

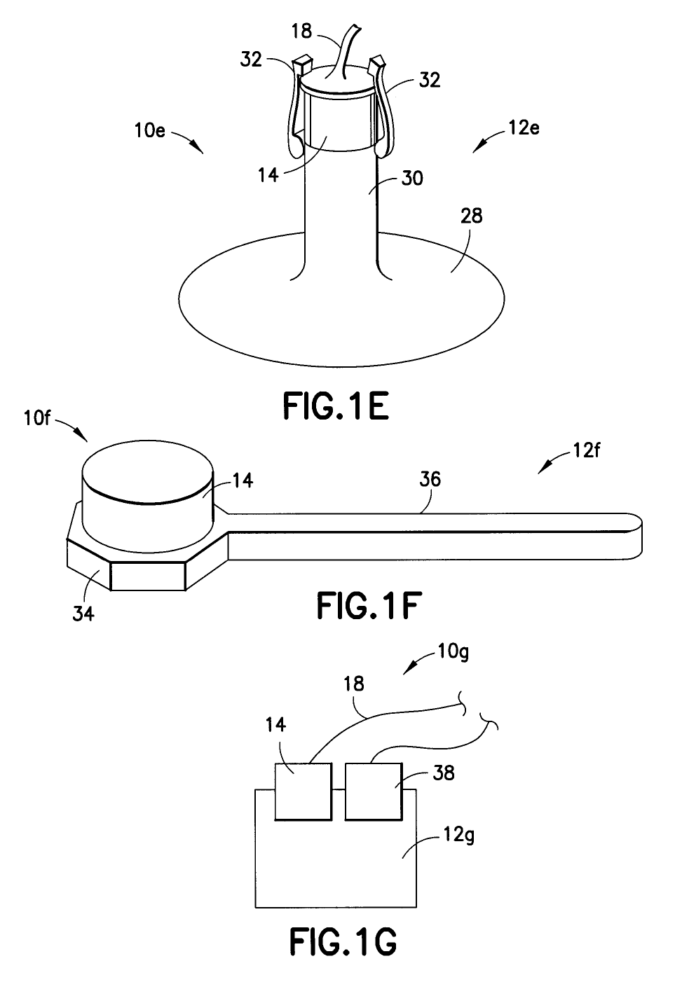

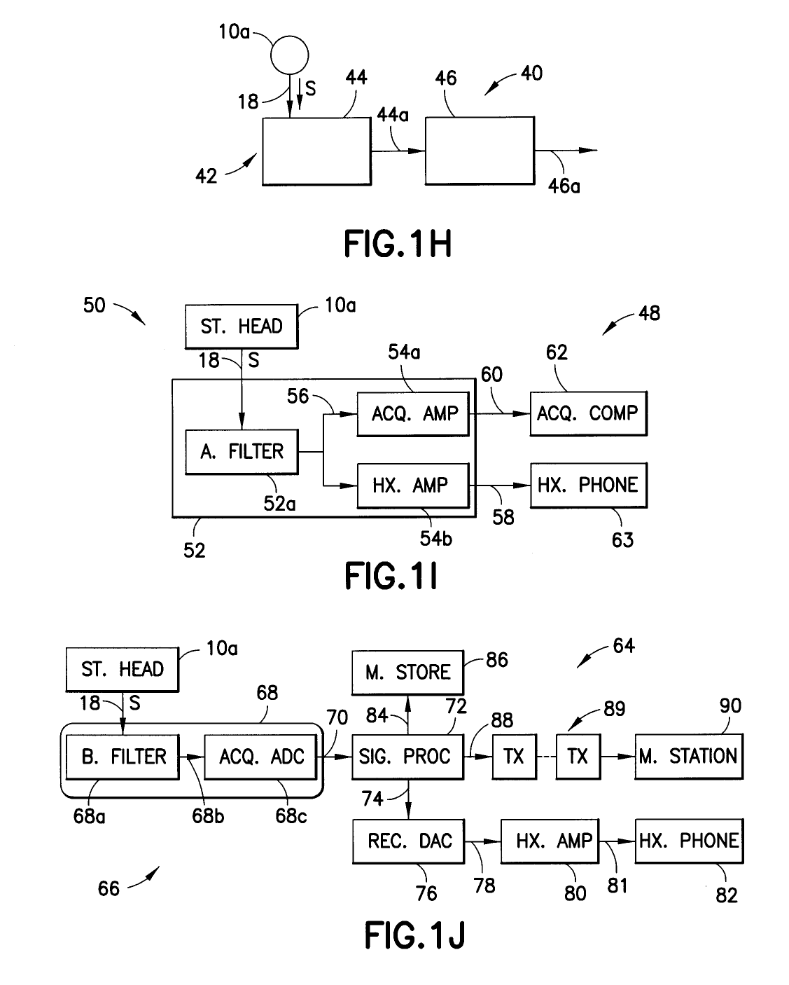

In one embodiment, a stethoscope head indicated generally at 10a in FIG. 1A comprises a head member 12a in which a transducer 14 is mounted. The head member 12a has a contact surface 16 which is applied to the body of a patient. The head member 12a is configured to detect and transmit bodily sounds from the patient in the area of contact. While the stethoscope head 10a (as well as other stethoscope heads disclosed herein) is described as being applied to the patient's body, the invention is not limited in this regard, and in other embodiments a stethoscope head 10a may be applied or attached to the patient's clothes. The transducer 14 detects the bodily sounds and converts the sounds from an acoustic form into electrical transducer signals which are conveyed via the transducer leads 18 to a processing system described below. The transducer 14 may be of any suitable type, including any type of microphone, e.g., a high-power microphone, an electret microphone, a condenser microphone, a dynamic speaker, or a piezoelectric vibration sensor, or the like. In other embodiments, the transducer may be equipped to transmit/convey transducer signals wirelessly instead of, or in addition to, using the transducer leads 18. In still other embodiments, the transducer leads 18 may be joined to other, pre-existing wiring such as a wire harness, which can be connected to a signal processor as described herein, so that the transducer signal is transmitted along with other signals on the pre-existing wiring connection. Optionally, the electronic stethoscope head 10a includes a plastic or other type of washable cover (not shown).

In various embodiments of the invention, a stethoscope head applied to a first location on a patient's body may be used in conjunction with one or more electrodes placed on the patient's body. In one embodiment, the one or more electrodes communicate directly with the electronic processing system, but in other embodiments, the electrodes may communicate with separate electronic processing systems which may provide an output signal which may be communicated to the electronic processing system described herein.

In one embodiment, the stethoscope head 10a includes an optional fastener 20 such as, for example, an adhesive applied to the contact surface 16, or a strap or tie, for securing the stethoscope head 10a to the patient's body. In some embodiments, the electronic stethoscope head 10a may remain applied or attached to the patient for extended periods of time and, compared to the patient and to prior art stethoscope heads, is relatively small in size. For an infant, the stethoscope head 10a may have a diameter in a range of, for example, about 15 millimeters to about 30 millimeters. To allow for long-term patient contact, yet prevent injury to patient's skin and particularly an infant's skin, some embodiments of the stethoscope heads 10a incorporate a specialty adhesive such as, for example, that currently used for ECG leads and thermal reflective patches sold under a Klear-Trace® brand electrodes by CAS Medical Systems, Inc. (Branford, Conn. USA). Such adhesives are currently used in NICU's to attach other sensors to infants. However, the present invention is not limited to medical grade adhesives, as other adhesives or attachment methods known to those skilled in the art, e.g., the use of adhesive tape, can be employed without departing from the broader aspects of the present invention.

In one embodiment, illustrated in FIG. 1B, a stethoscope head 10b comprises a head member 12b in the form of a bell, in which the transducer 14 is mounted.

In another embodiment, illustrated in FIG. 1C, an electrode is disposed between the stethoscope head 10c and the patient to avoid the need for the stethoscope head 10c to be in direct contact with the patient. As shown in FIG. 1C, the stethoscope head 10c comprises a head member 12c in the form of a diaphragm head 22 on which a diaphragm 22a is mounted. The stethoscope head 10c includes an optional electrode 24 on the diaphragm 22a. In one embodiment, the electrode 24 is of the type used to obtain EEG (electroencephalography), ECG (electrocardiogram), Pulse Oximetry, or other medical data. The electrode 24 includes an adhesive to retain the electrode 24 on the diaphragm 22a and, thereby, to retain the stethoscope head 10c on the patient.

In another embodiment, a stethoscope head 10d includes, as shown in FIG. 1D, a head member 12d in the form of a cup having a diaphragm 26 mounted thereon. The transducer 14 and connecting leads 18 are inserted in or through a portion (e.g., a base) of the head member 12d. The head member 12d is made from a stiff yet light-weight medical grade plastic such as, for example, polyphenylene. The contact surface 16 of the stethoscope head 10d is provided by a diaphragm 26 which seals the cup and, in one embodiment, has a coat of adhesive material (unnumbered) on its outside surface. An optional pin-hole 13 in the head member 12d neutralizes the inside air pressure with that of the ambient. The size of the pin-hole 13 may be, for example, about 0.35 mm in diameter or less, so as to not significantly affect the low-frequency response of the stethoscope head 10d.

According to still another embodiment, an electronic stethoscope head 10e, as seen in FIG. 1E, may comprise a head member 12e and the transducer 14 which are configured so that the transducer 14 is removably coupled or received by the head member 12e. This permits the head member 12e to be constructed from a relatively inexpensive material and to be disposed of after use on a patient, while the transducer 14 may be sanitized and coupled to a next head member 12e for subsequent use. For example, the head member 12e may comprises a base portion 28 for contact with the patient and a sleeve portion 30 which extends from the base portion 28 and which is sized to receive the transducer 14 by, for example, a press fit, threaded, or like connection. In one embodiment, the head member 12e may include releasable clips 32 thereon to help retain the transducer 14 in contact with the sleeve portion 30.

According to still another embodiment, an electronic stethoscope head 10f, as seen in FIG. 1F, may comprise the transducer 14 coupled to a head member 12f which is configured to have a paddle portion 34 and a wand portion 36. The paddle portion 34 provides a site for carrying the transducer 14 and the wand portion 36 allows for manipulation of the stethoscope head 10f for application of the stethoscope head onto hard-to-reach sites on the patient's body. The stethoscope wand 36 may optionally be used to position the transducer 14 in a patient's body, e.g., within the patient's abdomen.

As seen in FIG. 1G, a stethoscope head 10g may include a microphone 38 positioned on a head member 12g in addition to the transducer 14. The microphone 38 senses ambient noise during the use of the stethoscope head 10g. The ambient noise signal from the microphone 38 can be used to enable a noise-cancellation process on the signal received from the transducer 14 to provide a signal that more accurately reflects the patient's bodily sounds.

In one aspect of the invention, illustrated in FIG. 1H, the stethoscope head 10a (FIG. 1A) is operatively coupled to an electronic processing system 40 to provide, together, an electronic stethoscope system 42. The leads 18 from the stethoscope head 10a are connected to the electronic processing system 40, which includes an analog processing circuit 44 and a signal acquisition system 46. It should be appreciated that the stethoscope head 10a as shown in FIG. 1H is representative of all embodiments of stethoscope heads (10b-10g) described by the present description, any of which may communicate with the electronic processing system except where reference is made, expressly or implicitly, to one or more particular stethoscope heads. Similarly, the leads 18 should be taken to represent no only a direct wire connection, but also a harnessed or multiplexed connection as well as a wireless connection, except where one or the other of wire leads or a wireless connection is specifically required. The leads 18 transmit or convey a transducer signal S derived from the bodily sounds of the patient to the electronic processing system 40. At the electronic processing system 40, the conditioning circuit 44 receives the transducer signal S, conditions the transducer signal S and provides a conditioned signal 44a for use by the signal acquisition system 46. In one embodiment, conditioning may include, for example, amplifying the transducer signal S and/or filtering the transducer signal S to reduce or prevent aliasing in a subsequent sampling process in the signal acquisition system 46. For example, to avoid aliasing, a filter with a cutoff of about 5 kHz or greater may be applied by the electronic processing system 40. The signal acquisition system 46 is configured to digitize the conditioned signal 44a using a sampling process and to use, for example, a DTSP filtering technique to apply a low-pass filter to the digitized transducer signal with a cut-off of about 1.6 kHz or less, e.g., a cut-off of about 800 Hz or less, for example, a cut-off of about 300 Hz or even about 200 Hz. By digitizing and filtering the conditioned signal 44a (e.g., the processed transducer signal S), the signal acquisition system 46 generates a digital output signal 46a which includes the original transducer signal S in digital form. The output signal 46a can be visually displayed, further analyzed, stored or recorded, and as described below compared to one or more reference signal profiles to assist in diagnosing the patient. For example, in one embodiment, the digitized signal 46a is used to determine the condition of a patient and whether an intervention is needed, as described herein.

Optionally, various components in the electronic processing system 40 can be programmable and/or re-programmable, to allow for updates, corrections, self-tests, calibration, and repair. In one embodiment, the processing system may be configured so that programming and/or reprogramming can be performed remotely. In another embodiment, the programming, reprogramming and/or programming upgrade can be provided by means of a platform flash drive.

Each electrical component of the electronic processing system 40 as disclosed herein may communicate with other components as described herein either by hardwire or cable connections, or wireless communication connections.

It should be appreciated that the electronic stethoscope head 10a (as well as heads 10b-10g), the electrical processing system 40, and the electronic stethoscope system 42 as described herein, allow medical practitioners and personnel, healthcare workers, and like caregivers, to apply the stethoscope head 10a to a patient and to monitor the transducer signals S from the stethoscope head from a remote location. Remote monitoring may include conveying some signals via a telephone system, by radio waves and/or over an intranet or internet. The features disclosed herein also allow for convenient off-site collection and storage of crucial data obtained from the stethoscope heads. Storing signal data permits later analysis. Another advantage of the electronic stethoscope head 10a, the electronic processing system 40, and the electronic stethoscope system 42 as described herein is the ability to detect, record, analyze and process, in electronic form, bodily sounds that would otherwise be undetectable to a person using conventional acoustic stethoscopes, including sounds whose magnitudes are too small to be perceived and sounds whose frequencies are outside the human audible range. The electronic processing system 40 and electronic stethoscope system 42 disclosed herein can be configured to detect, record, analyze and process such sounds and to present the results to a caregiver in various ways, including graphically, e.g., by means of a video display, and audibly, e.g., by altering the amplitude or shifting the frequency of the sounds into audible range, and with and without comparison to reference or threshold signals and data.

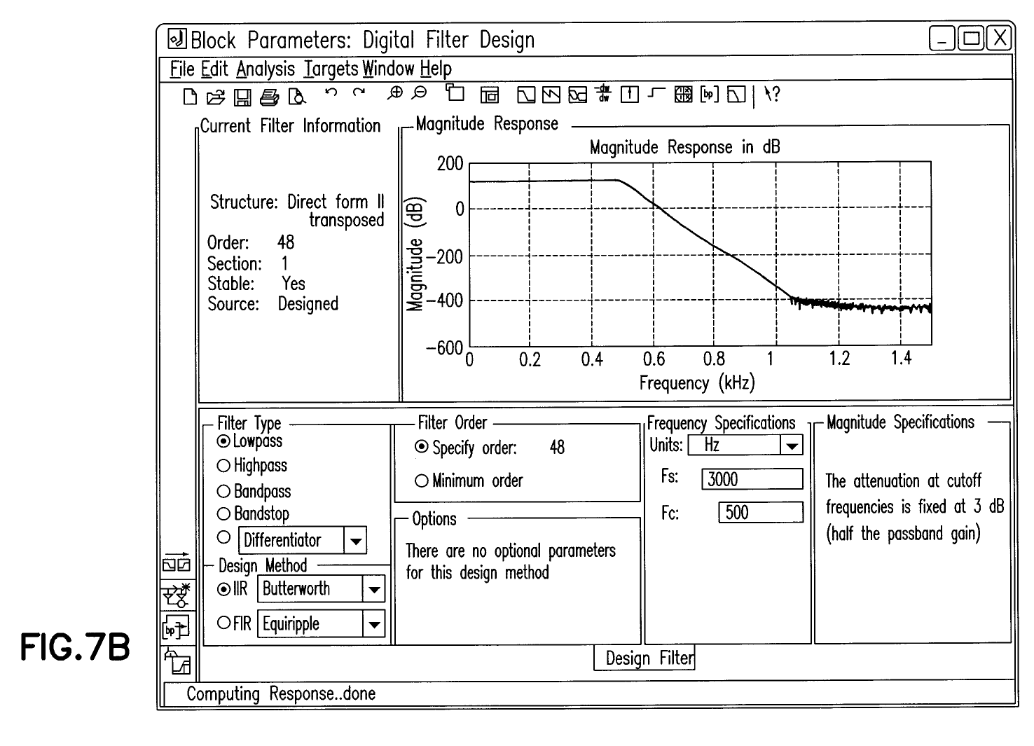

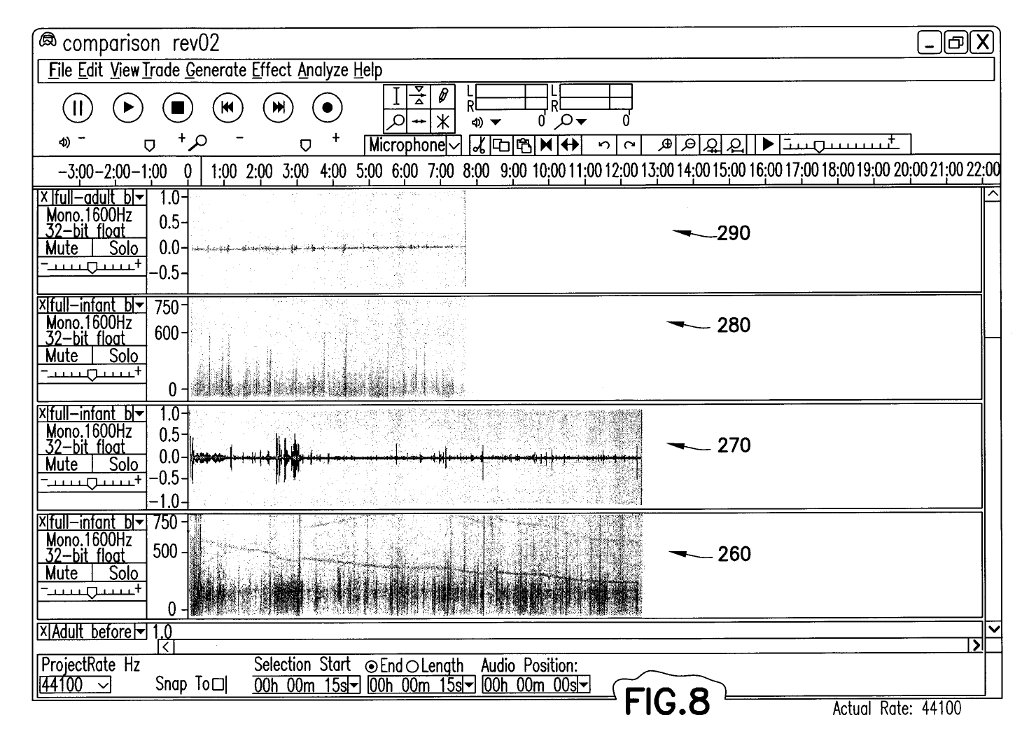

In one embodiment of the invention, an electronic stethoscope system 48 shown in FIG. 1I includes an electronic processing system 50 which comprises conditioning circuitry 52. The conditioning circuitry 52 includes an optional first stage (A.Filter) 52a which receives and filters the transducer signal S passed by the leads 18 from the stethoscope head 10a. The first stage (A.Filter) 52a is a second order low-pass filter such as, for example, a Butterworth filter (FIGS. 7A and 7B) with a cutoff frequency of approximately 1 kHz. The first stage (A.filter) 52a produces a filtered transducer signal 56 from the transducer signal S and emits the filtered transducer signal 56 to a first amplifier circuit (Acq. Amp) 54a. The first amplifier circuit (Acq. Amp) 54a produces a conditioned signal 60 for use by a signal acquisition system (Acq.comp) 62. Overall, the first stage (A.filter) 52a and the first amplifier circuit (Acq. Amp) 54a provide a signal having a variable gain from about 20 to 200. An optional headphone amplifier (Hx.amp) 54b (which may be similar to the first amplifier circuit (Acq. Amp) 54a) receives the quasi-conditioned transducer signal 56 and generates an analog output signal 58 which is suitable for driving a small acoustic output device 63 such as, for example, a speaker or headphones. In one embodiment, the analog output signal 58 is a signal of about 100 milliwatts or less.

In one embodiment, the signal acquisition system (Acq.comp) 62 is a general purpose personal computer equipped with a sound board which includes an analog to digital converter and which is programmed with software capable of converting the signal 60 to digital form, applying the low-pass filtering described herein using DTSP techniques, and storing the signal. Such software includes, but is not limited to, LABVIEW®, sold by National Instruments Corporation (Austin, Tex.), AUDACITY® (developed by Dominic Mazzoni (South Pasadena, Calif.), MATLAB® scripts written in MATLAB® branded software sold by MathWorks Inc. (Natick, Mass. USA), Octave scripts, Wave Surfer, and Python scripts (using Python software, distributed by Python Software Foundation (Hampton, N.H.). Programs such as MATLAB®, SIMULINK® (sold by MathWorks, Inc., (Natick, Mass.), and AUDACITY® provide the ability to filter and visualize bowel sound signal and similar signals. AUDACITY®, for example, has a simple point-and-click interface to provide well known filtering techniques such as low-pass filtering.

In another embodiment, an output signal from a conventional electronic stethoscope (not shown) can be transmitted to the signal acquisition system (Acq.comp) 62 for processing as described herein.

In another embodiment, illustrated in FIG. 1J, the transducer leads 18 from the stethoscope head 10a are connected an electronic stethoscope system shown generally at 64, which includes an electronic processing system 66 and a conditioning circuitry 68. The conditioning circuitry 68 includes a first stage processor (B.Filter) 68a. The first stage processor (B.Filter) 68a comprises an amplifier and low-pass filter which generates a filtered signal 68b. The filtered signal 68b is provided to an analog to digital converter (Acq.ADC) 68c, which generates a digital signal 70. The first stage processor (B.Filter) 68a provides filtering to remove out-of-band signals, not suitable for Nyquist sampling by the acquisition digital to analog converter (Acq.ADC) 68b. The first stage processor (B.Filter) 68a also provides automatic gain control to maximize the dynamic range of the analog to digital converter (Acq.ADC) 68c. The digital signal 70 is provided to a signal processor 72 which performs discrete time signal processing (DTSP) and generates output signals. The DTSP techniques provide further filtering in a manner that is more flexible than non-digital circuitry provides. In being performed numerically, DTSP techniques can be performed by various devices including by not limited to digital signal processor (DSP) chips, field programmable gate array (FPGA) chips, as well as personal computers. An FPGA is an array of configurable logic blocks along with a configurable interconnecting resource, sometimes referred to as a FPGA fabric. An output signal 74 is provided to a digital to analog converter and reconstruction filter (Rec.DAC) 76 which yields a reconstructed analog signal 78 which is provided to an amplifier (Hx.Amp) 80. The amplifier (Hx.Amp) 80 provides an output signal 81, typically less than 100 milliwatts for driving, for example, conventional output device 82 such as headphones (Hx.Phone) or a display device. The signal processor 72 may also provide an output signal 84 to an optional memory device (M.Store) 86, which may comprise an SD card or Compact FLASH device or the like, for storage and later retrieval. In one embodiment, the signal processor 72 communicates, for example, by a transmitter-receiver combination shown generally at 89, an output signal 88 to a remote monitor station (M.station) 90, which may comprise a general purpose computer programmed to monitor and exhibit the output signal 88.

It should be appreciated that the electronic processing systems 40, 50 and 66 as described herein, perform discrete time signal processing which is not limited to the following descriptions. Signal processing may include, for example, de-noising techniques to remove interfering signals and noise inherent in the environment. Enhancement of the conditioned output signals 60, 81 cause a signal to be more prominent with respect to other signals. Techniques such as, for example, frequency shifting and/or frequency stretching may be applied so that the medical personnel, healthcare worker, or caregiver can better interpret the signals 60, 81. For example, when a range of signal frequencies is partially out of the range of hearing with a conventional stethoscope, frequency shifting moves the signal frequency range to a range that is fully audible by the human ear. As described herein, signal processing may include filtering and/or comparing the output signals 60, 81 to a baseline or reference signal. In one embodiment, the baseline or reference signal or signals may include known signal ranges and/or thresholds determined by medical personnel, healthcare practitioners and the like, to be indicative of healthy or otherwise acceptable bodily sounds. In one embodiment, the baseline signals are exhibited (e.g., on a display device) in proximity to (e.g., a split screen comparison) real-time detected and processed signals representing bodily sounds of a patient undergoing diagnosis so that the medical personnel can compare detected signals to the reference to aid a more rapid and more accurate diagnosis. In one embodiment, signal processing includes use of a lowpass filter to filter out frequencies between about 250 Hz and about 400 Hz and a highpass filter to filter out frequencies between about 50 Hz and about 100 Hz.

In one embodiment, the system is configured to filter out speech by the patient or by those around the patient that may be picked up by the stethoscope head. Optionally, the patient and/or medical personnel, healthcare worker, or caregiver, may be provided with a switch to activate the speech filter, to protect the privacy of selected conversations from detection by the transducer 10 and recordation by the processing system.

Optionally, the transducer signal S data, electronic data generated by the electronic processing system 66 as described herein, and/or the reference or baseline signals are stored in memory resident in the signal processor (Sig.Proc.) 72 or in a memory device (M.Store) 86 in communication with the signal processor 72. Data is stored in such a memory device using a common file system not limited to, for example, the FAT file system or a journaling file system such as LogFS. Such a memory device will have data accessible using common access devices. The processor as well as any displays employed in the embodiments described herein can be battery driven, powered vie electricity from a wall-type receptacle, or by a combination thereof.

In some embodiments of the invention, the signal processor 72 performs wavelet based analysis or Fourier based analysis. Unlike Fourier analysis, wavelet analysis is known to localize in the frequency and time domains, enhancing the ability to detect explosive peaks like those present in bowel sounds which are indicative of problematic conditions. As should be appreciated, in one embodiment, the signal processor 72 stores or is provided the reference or baseline signals for signal comparison between the output signal 84 and the baseline signal. Optionally, the signal processor 72 includes programmed routines having a set of rules which are used to activate audio and/or visual alarms if the conditioned output signal is detected above or below a predetermined threshold which is indicative of an unhealthy or unacceptable detected bodily sound. It should be appreciated that such rules are not limited to the detection of abnormal signals, but also equipment problems and/or to alert or advise medical personnel, healthcare practitioners, caregivers and the like, to cautionary events.

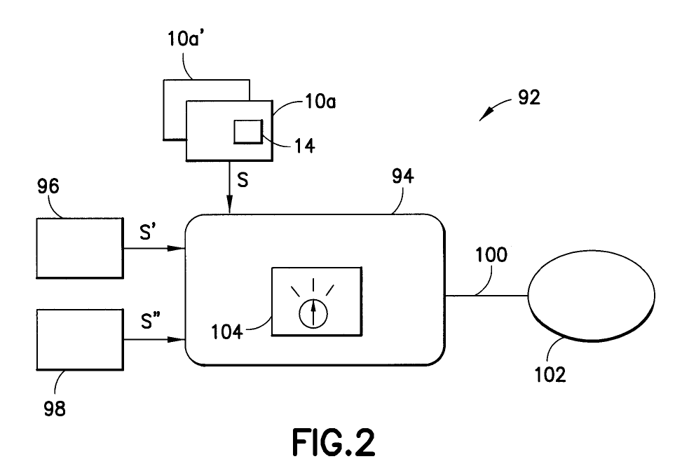

Optionally, an electronic processing system 40 (i.e., a conditioning circuit and a signal processor system) as described herein may be configured to receive and process signals from one or more stethoscope heads and/or electrodes. For example, the multi-lead electronic stethoscope system 92 shown in FIG. 2 includes a processing system 94 configured to receive the transducer signal S from the transducer 14 in the stethoscope head 10a and to receive a second transducer signal S′ from a second device 96 and, optionally a third transducer signal S″ for a third device 98, and to generate an output signal 100 to an output device 102 for presenting the output signal 100 to medical personnel, a healthcare practitioner, or like caregiver, operating the system 92, or for storage and future processing. The second device 96 may be, for example, a noise-cancellation microphone in the stethoscope head 10a; and the third device 98 may be an ECG electrode affixed to the patient. Other signals that may be received and processed by the multi-lead electronic stethoscope system 92 may include one or more stethoscope heads 10a, 10a′ for monitoring, for example, right and left lung sounds independently; a combination of heart and bowel sounds, etc., and/or for providing such sounds in mono, stereo or triphonic sound. Optionally, the multi-lead electronic stethoscope system 92 may include a channel selector switch 104 for a user, whereby the user can select a particular signal of interest to be displayed, analyzed or reproduced in acoustic form via a headphone or other speaker.

In another embodiment, the electronic stethoscope system 42 may be configured to control ancillary devices in response to conditions indicated by signals S, S′, S″ from the stethoscope head 10a and/or other devices. For example, the electronic stethoscope system 42 may be configured to activate a vibrotactile stimulator (not shown) to stimulate a patient, for example, an infant, to breathe, if the infant's respiration, heart sounds, and/or, possibly, an ECG signal, indicate that such stimulus is needed or desired. Optionally, such a vibrotactile stimulator can be run wirelessly and can be coupled to the stethoscope head 10a, embedded in the same adhesive as the stethoscope head 10a, or separate fastening system. Similarly, an electronic stethoscope system 42 may be configured to control an automatic feeding pump or automatic suction pumps for bowel obstructive disorders (not shown), should a patient's bowel sounds indicate a need or desire for such treatments.

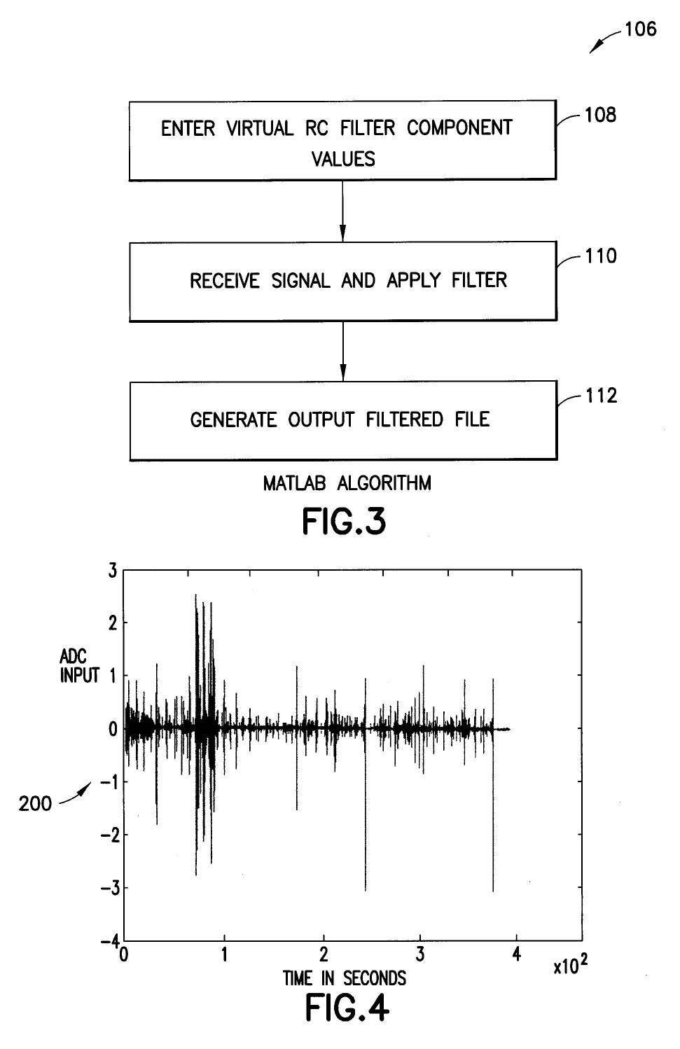

In one embodiment, the signal acquisition system (Acq.comp) 62 is implemented in a personal computer and includes a low pass filter. For example, the signal acquisition system (Acq.comp) 62 includes a model-based simulator such as a MATLAB® branded software filter. The MATLAB® system filters the data in the data files to reduce or substantially eliminate frequencies above, for example, about 200 Hz, produces a realistic roll-off, maintains the sounds needed for analysis and comparison, and yields filtered audio files in addition to the unfiltered audio files. An illustrative example of an algorithm for the low pass filter is indicated generally at 106 in FIG. 3. In step 108, the user enters the relevant filter parameters for the filter. In step 110, the signal is received and the filter is applied thereto. In step 112, an output file is generated and stored.

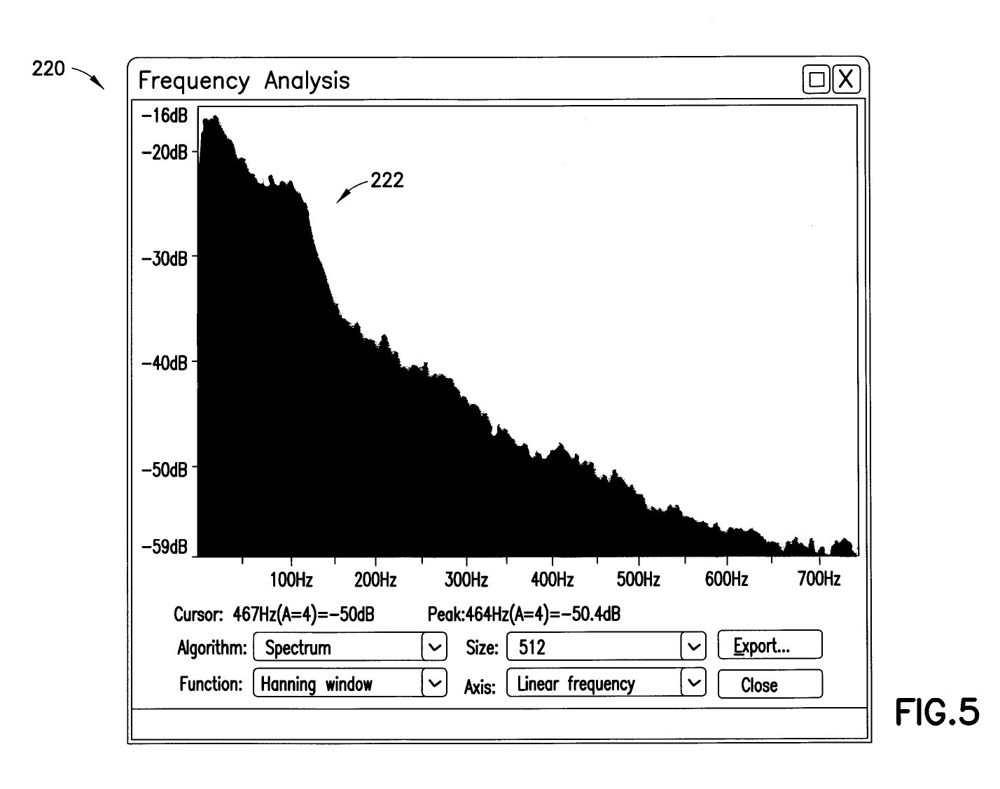

FIG. 4 depicts a sonograph 200 of example bowel sounds of an infant detected and recorded before feeding. The sonograph 200 generally illustrates the output 58 of the electronic stethoscope system 48 of FIG. 1I and/or the output 81 of the electronic stethoscope system 64 of FIG. 1J. To an untrained ear such a recording sounds like popping. To detect such sounds, the electronic stethoscope head 10a is held against a patient to minimize introduction of additional acoustic artifacts.

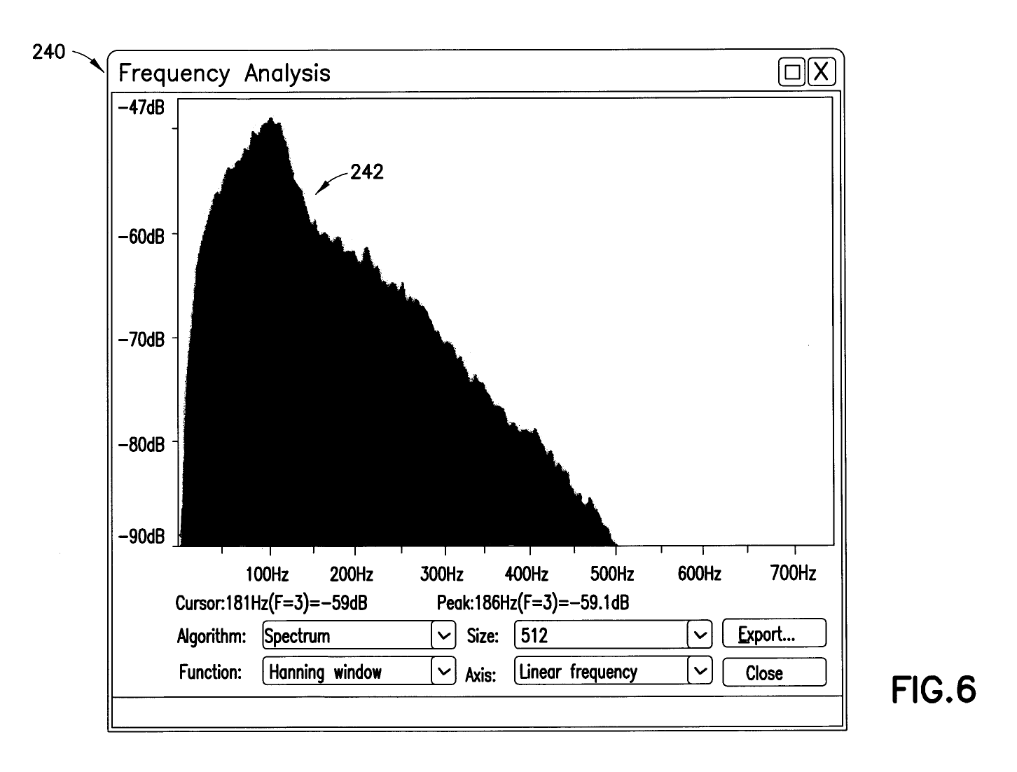

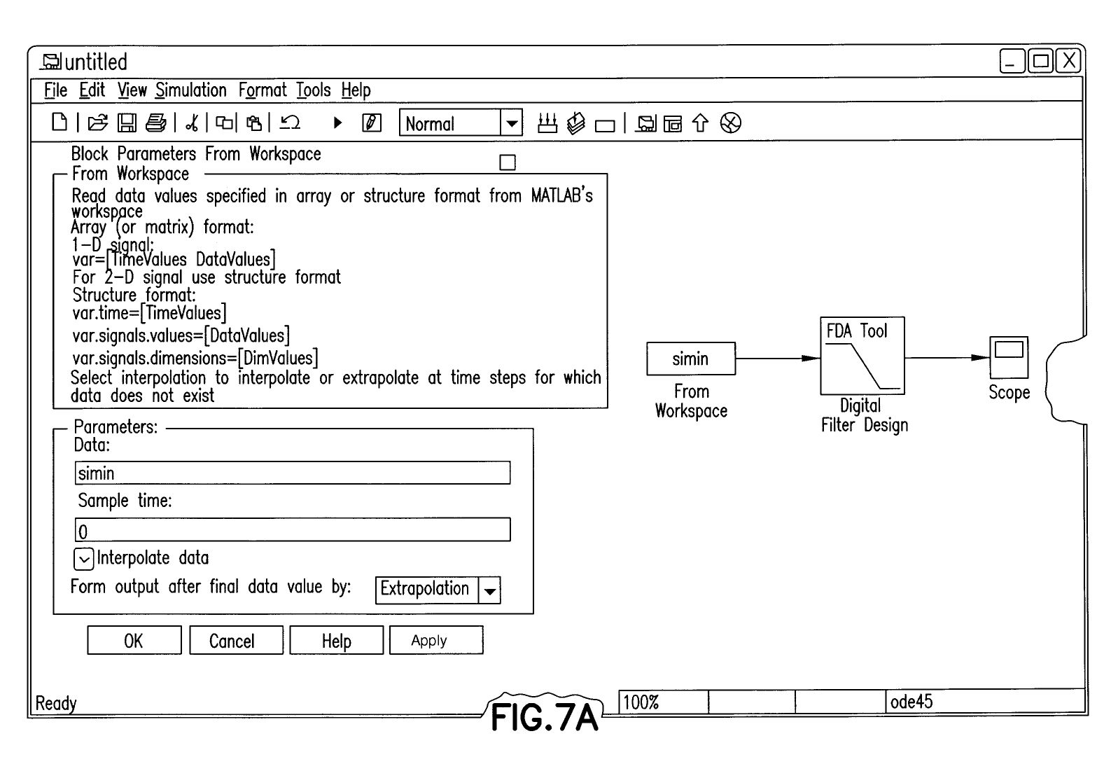

FIG. 5 and FIG. 6 illustrate spectra 220 and 240, respectively, before and after filtering, as exhibited by a display feature of an audio editing program such as, for example, an open source software program branded AUDACITY®. Once the data is imported into the AUDACITY® system, the waveform is analyzed by examining its plot spectrum (e.g., spectra 220 and 240) represented in FIG. 5 and FIG. 6. The highest peak areas shown generally at 222 and 242, respectively, in the spectrum 220 and 240 indicate the frequencies of most of the recorded sounds. As shown in FIGS. 5 and 6, the inventors have discovered that bowel sounds produce a sharp spike in the waveform and are found to be the loudest noises in the recordings. Once the range of most of the sounds is determined according to the frequency plot spectrum, a low pass filter is run. The low pass filter reduces the background noise and isolates the bowel sounds for a more audible signal for comparison, analysis and diagnosis. In one embodiment, a low pass filter may be run at about 300 Hz with a filter quality of about 0.7 per 6 dB per octave rolloff, e.g., 0.7071 per 6 dB per octave rolloff. Applying the high pass filter attenuates frequencies above 300 Hz. The higher the rolloff value the more frequencies above the cutoff value are attenuated. Applying a filter quality of above 0.7071 increases the resonance of the cutoff frequency and could result in clipping, which is undesirable. It should be appreciated that the invention is not limited to the cutoff and rolloff values stated here, and in other embodiments, other values for the cut-off frequencies and filter quality may be used.

A comparison of the two categories of sound recordings made of bowel sounds of healthy adults and infants, i.e., before and after eating, were rendered in graphic form as shown in FIG. 8 using the AUDACITY® software. Before eating, bowel sounds are notably more pronounced as the stomach and intestines are very active before digestion. For example, graph 260 illustrates sound detected, recorded and exhibited in an adult before eating, and graph 280 illustrates sound detected, recorded and exhibited in an infant before eating. By comparison, after eating, the bowel sounds of both adults and infants decrease steadily until almost no sounds are heard. For example, graph 270 illustrates sound detected, recorded and exhibited in an adult after eating, and graph 290 illustrates sound detected, recorded and exhibited in an infant after eating. As shown in the graphics of FIG. 8, the detected, recorded and exhibited sound of both the adult and the infant subjects exhibit similar peak patterns.

In one embodiment, the electronic stethoscope system 42 includes the transducer 14 which is an electret or condenser microphone, and such a transducer may be connected to a microphone input port of a computing device such as, for example, a laptop or personal computer running a Windows or Macintosh operating system. As described above, software including but not limited to the following are used to detect, record and graphically illustrate signals from such a port: AUDACITY®, WaveSurfer, and Realtime Analyzer from Yoshimasa Electronic Inc.

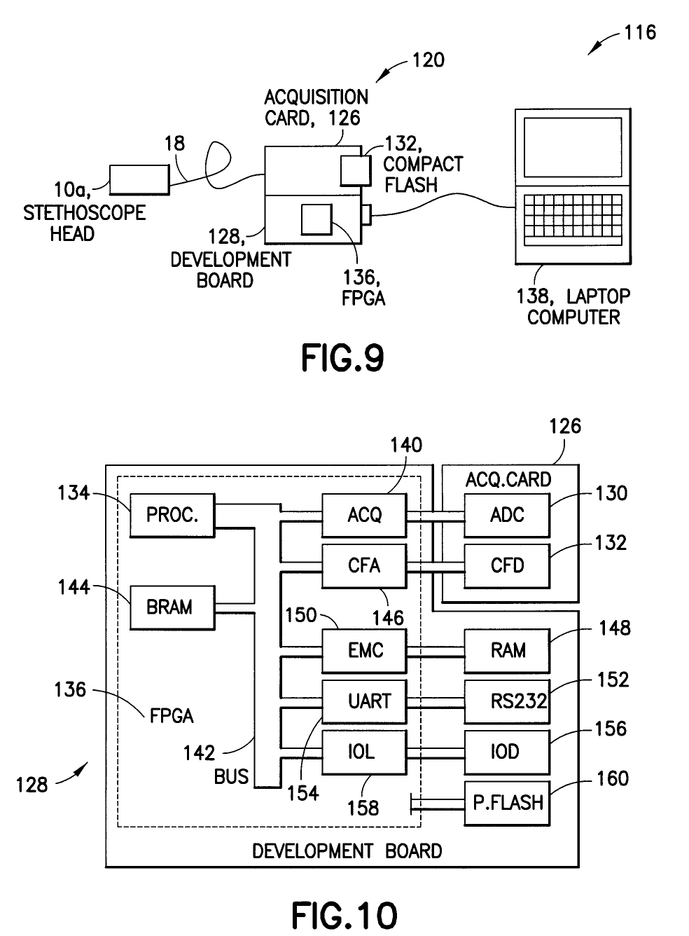

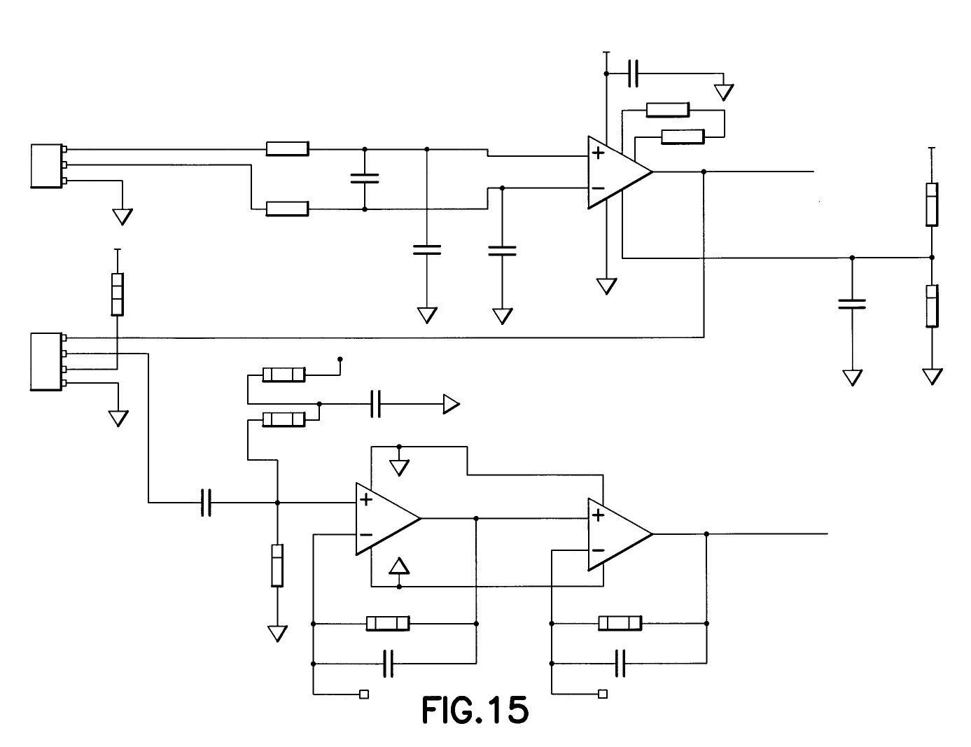

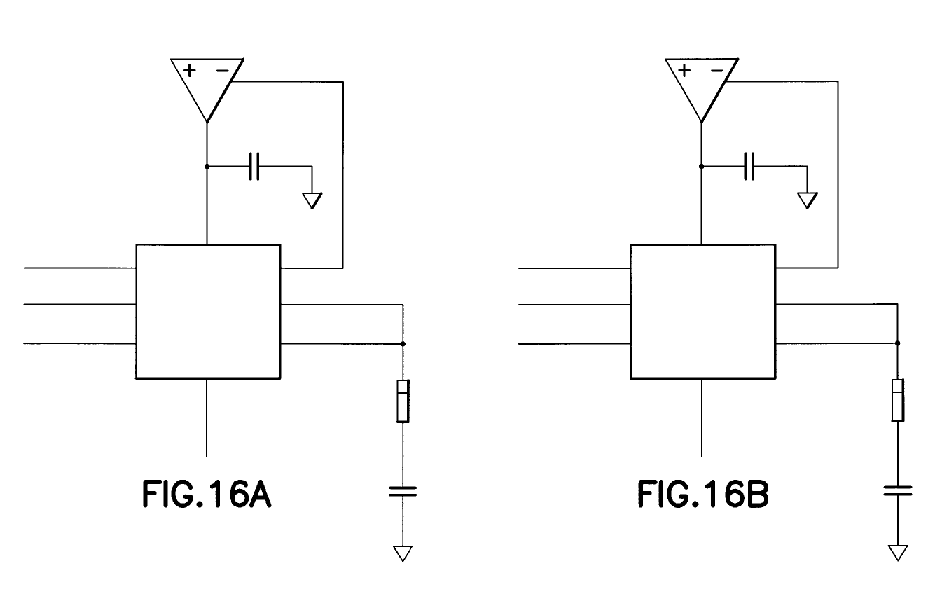

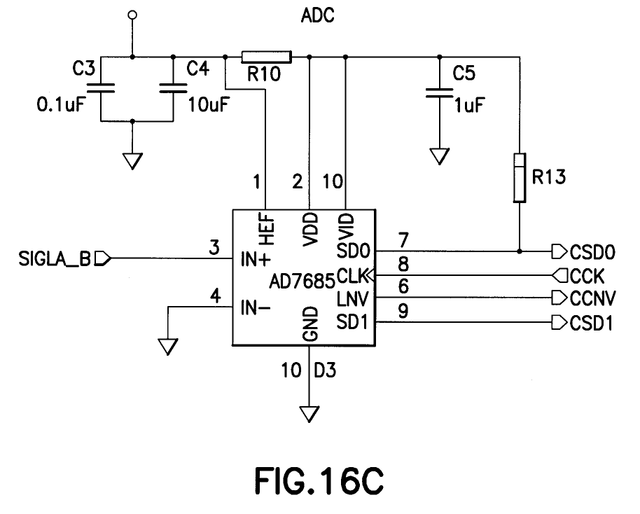

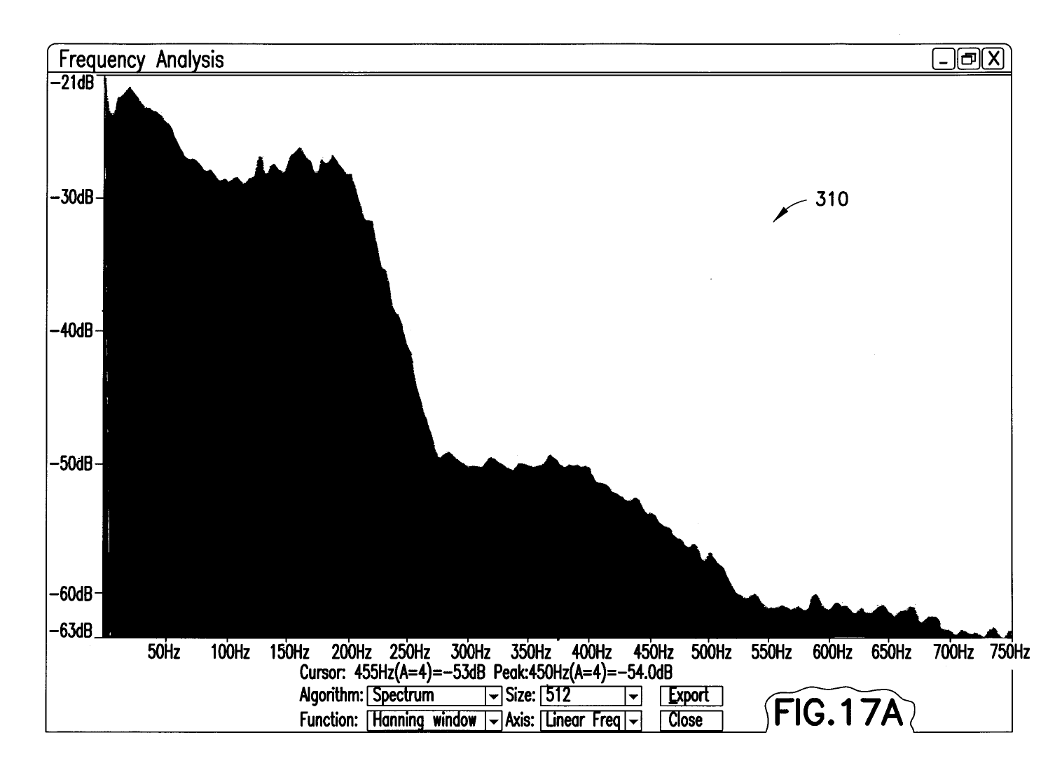

In still another embodiment, an electronic stethoscope system 116, shown in FIG. 9, includes the electronic stethoscope head 10a and an electronic processing system 120, part of which is also depicted in FIG. 10. Portions of the electronic processing system 120 are mounted on an acquisition card 126 and a development board 128, but the invention is not limited in this regard, and in other embodiments the components of the electronic processing system may be assembled in any convenient fashion. Parts of electronic stethoscope system 116 are also represented in FIG. 14, which shows an amplifier (also shown in FIG. 15,) potentiometers (also shown in FIG. 16A and FIG. 16B), an analog to digital converter (also shown in FIG. 16C), a connector for an FPGA board and for a compact flash device. The acquisition card 126 carries amplifiers as seen in FIG. 15, and an analog to digital converter (ADC) 130 (FIG. 10) used to sample and digitize the signal from the transducer 14. In one embodiment, the amplifier of FIG. 15 includes two amplifiers, including an AD627, which is an instrumentation amplifier that is optionally used with transducers that provide a differential signal output, and a switch connector SW1 which determines if the instrumentation amplifier is bypassed. The two operational amplifiers are used in the non-inverting amplifier configuration to provide a variable gain set by the digitally controlled potentiometers (FIG. 16A and FIG. 16B). Each amplifier includes a capacitor in the feedback path to roll off the gain at approximately 1.6 kHz.

In one embodiment, the ADC 130 may be an AD7685 16-bit, analog-to-digital converter which provides 16 bit samples at rates as high as 250 kHz. Sample rates are presently limited by the data rate of the associated communications. In one embodiment, the acquisition card 126 includes an amplifier circuit as seen in FIG. 15. To further address the issue of large dynamic range inherent with bowel sounds, the acquisition card 126 may include digitally controlled potentiometers of the type shown in FIG. 16A and FIG. 16B to maximize the dynamic range of the sampling function of the acquisition card. Input configurations are provided for differential as well as single ended signal sources.

The acquisition card 126 includes and communicates with a compact Flash memory card 132 via a flash card connecter, to provide long term stand-alone data collection capability. The acquisition card 126 is operably connected to the development board 128 by a card connecter.

The development board 128 includes a microprocessor system to provide discrete time signal processing and data management functions, such as managing a data acquisition system, communicating with other devices, and performing data logging. In one embodiment, the development board 128 includes an embedded microprocessor 134 in the development board 128 to enable the use of advanced signal processing algorithms such as, for example Fourier Transformations and the like. For example, the development board 128 may include a field programmable gate array (FPGA) 136 to implement the signal processor system. A suitably programmed general purpose computer 138 (e.g., a laptop personal computer) is used for data collection.

The ADC 130 is controlled by Acquisition logic (ACQ) 140. The processor 134 on the development board 128 connects through a bus 142 to the on-chip memory resource (BRAM) 144 and peripheral devices. The compact Flash device (CFD) 132 communicates with and is controlled by compact Flash adapter logic (CFA) 146 via the flash card connector. Additional memory resources (RAM) 148 are accessed via the external memory controller (EMC) 150. Serial communications devices (RS232) 152 are controlled by serial communications logic (UART) 154, but the invention is not limited in this regard, and in other embodiments, USB or equivalent serial communications can be used. Input-output devices (IOD) 156 such as push-buttons, switches, light emitting diodes, and seven-segment displays are controlled by input-output logic (IOL) 158. The platform Flash (P.Flash) 160 configures the FPGA device as required, following the application of reset. Code written with a hardware description language such as VHDL is used to produce an image file or bit file, stored in the platform Flash memory 160, which configures all aspects of the FPGA 136, including the on-chip memory (BRAM) 144. Once the FPGA 136 is configured, it executes machine code just like any processor. As such, the software is written using conventional software development tools, such as those used with the ‘C’ programming language.

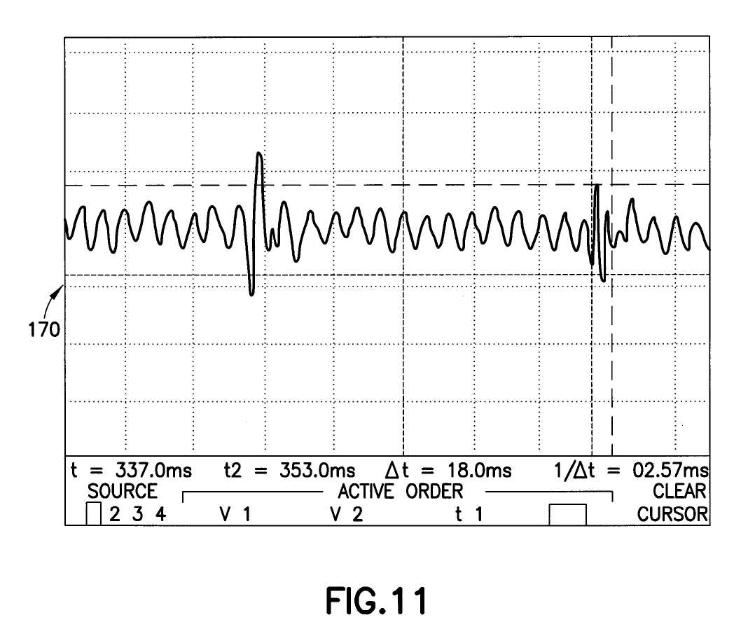

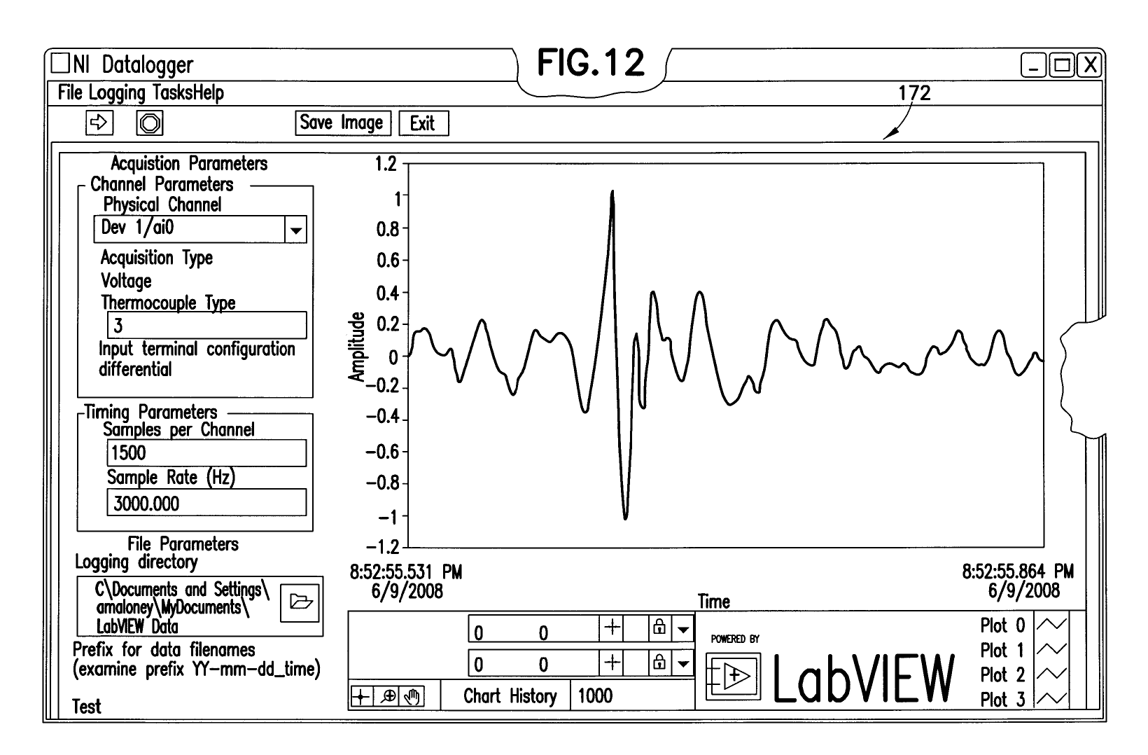

In one execution of the electronic stethoscope system 116, the heart beat sound of an adult volunteer was recorded and the analog signal 170, before sampling, was exhibited on a display device such as, for example, an oscilloscope, as shown in FIG. 11. The analog signal 170 was digitized by the electronic stethoscope system 116 and passed via the serial communications port (RS232) 152 to the personal computer 138 where the digitized signal was plotted 172 (FIG. 12) using LabVIEW® data acquisition and analysis software.

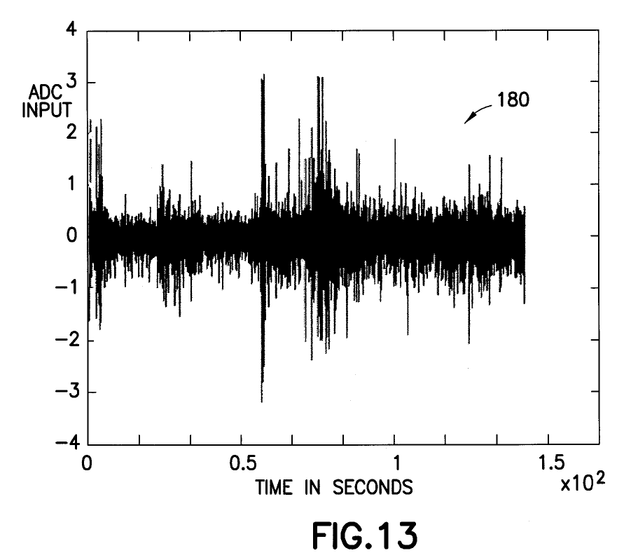

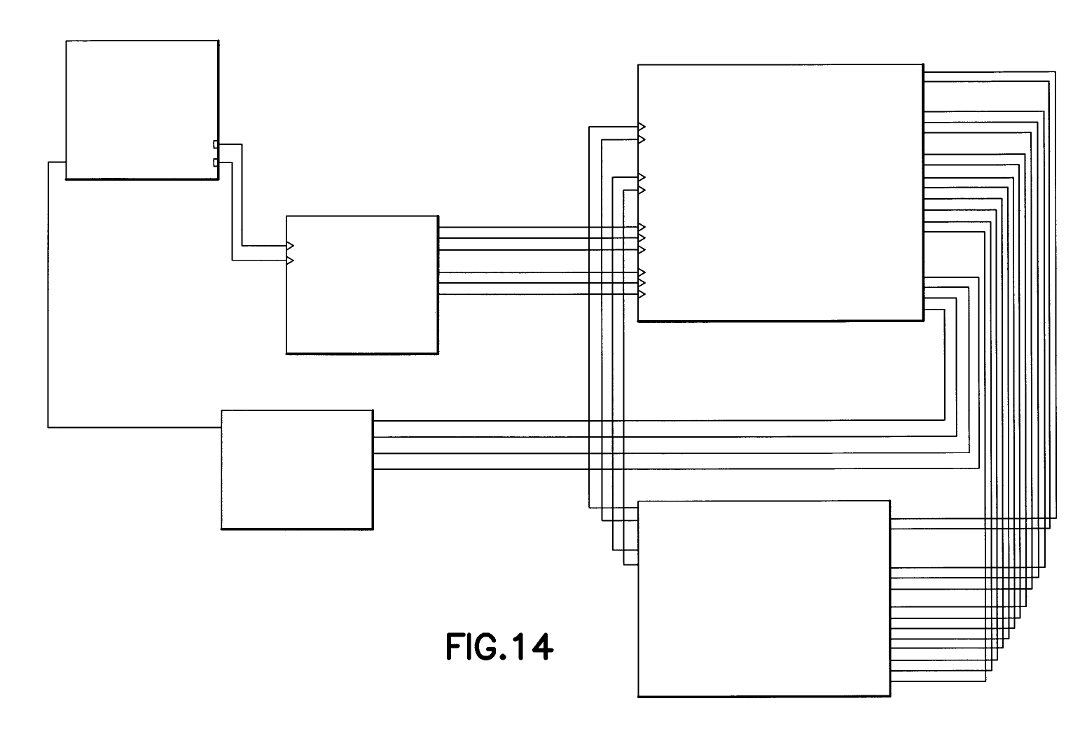

FIG. 13 shows digitized bowel sounds 180 from an adult produced by the electronic stethoscope system 116 which includes the electronic stethoscope head 10d and the acquisition card 126 and the development board 128, as exhibited by the MATLAB® software executing on the personal computer 138. As illustrated in FIG. 13, the digitized bowel sounds 180 were not filter prior to display. By comparing the digitized bowel sounds 180 of FIG. 13 to the sonograph 200 of FIG. 4 showing the bowel sounds of the infant, it can be seen that the plots share a similar pattern of peaks even though FIG. 13 shows that substantial background noise is in the waveform.

As is generally known, a conventional electronic stethoscope operates for a very limited time such as, for example, in two minute (2 min.) intervals due to, for example, a built-in automatic battery-saving turn-off timer. The stethoscope head is large and heavy and is not suitable to be attached to the abdomen of a premature infant for a period of time extending beyond an hour at a time. Such stethoscopes provide a low frequency response like that of the non-electronic stethoscope, unnecessarily limiting the low frequency cutoff, in some cases between 20 Hz and 100 Hz. Such electronic stethoscopes do not provide analysis capability, provide only simple filtering and like the Littman series of stethoscopes provide only passive noise cancellation ability. In addition, a conventional electronic stethoscope is for general use and may introduce selective distortion into a bowel sound signal.

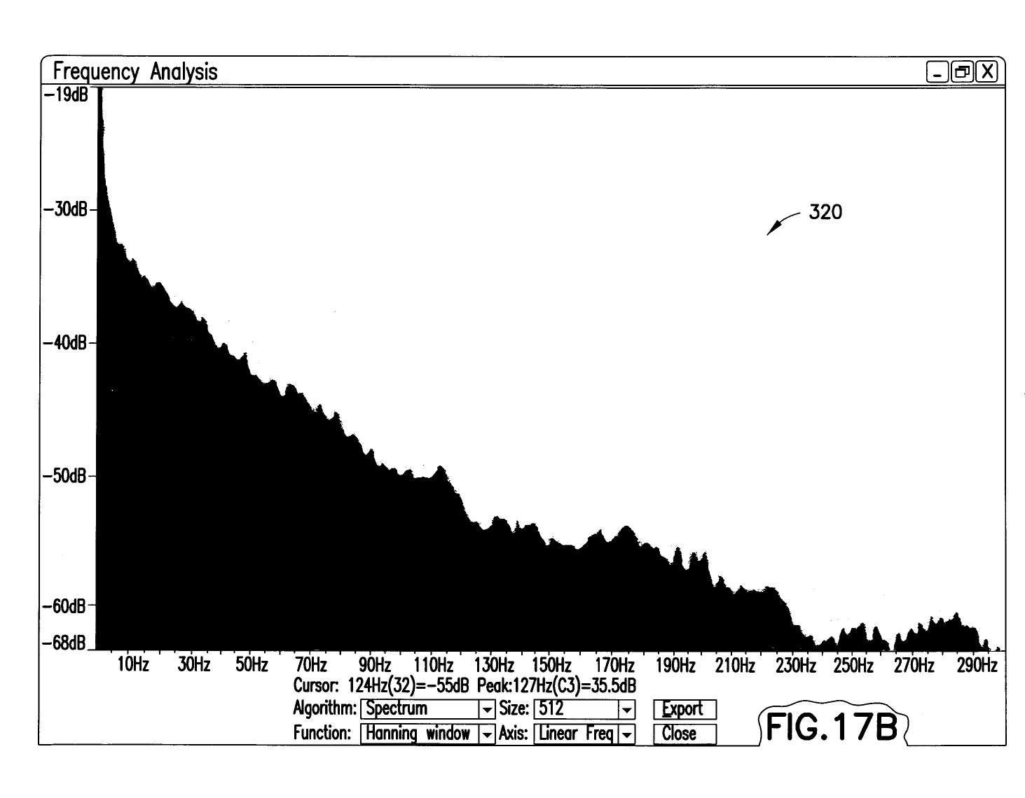

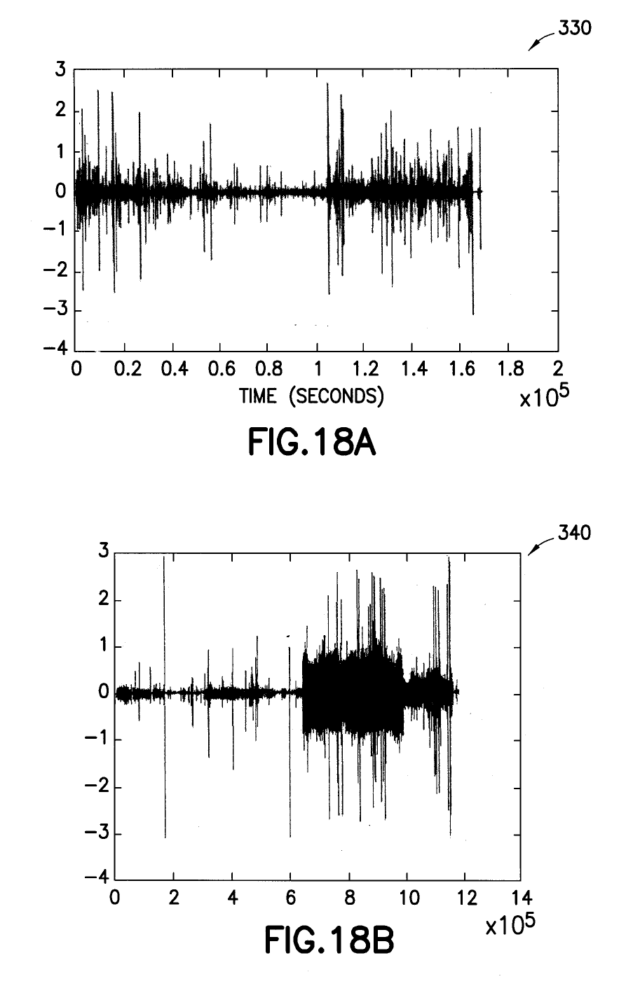

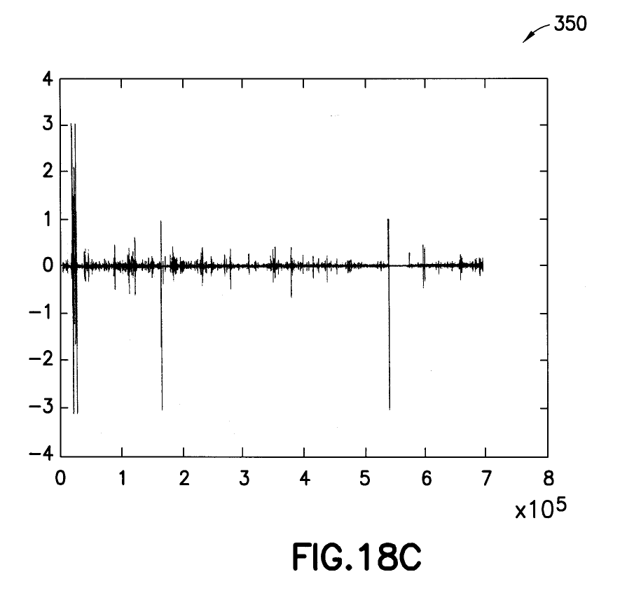

In one embodiment, an electronic stethoscope system as described herein was used to generate variety of sonographs. For example, FIG. 17A and FIG. 17B are monographs 310 and 320 of an infant's bowel sounds, before and after eating, respectively. The monographs 310 and 320 illustrate that infant bowel sounds after eating 320 occur with less frequency than bowel sounds before eating 310, and the bowel sounds drop off in intensity more quickly at higher frequency than bowel sounds before eating. Similarly, the electronic stethoscope system was used to generate the sonographs shown in FIG. 18A, FIG. 18B and FIG. 18C. FIG. 18A is a sonograph 330 of normal infant bowel sounds (with the horizontal axis indicating a time scale), FIG. 18B is a sonograph 340 of the bowel sounds of an infant with necrotizing enterocolitis, and FIG. 18C is a sonograph 350 of the bowel sounds of an infant with gastroesophageal reflux disease (GERD) (the horizontal axes of FIG. 18B and FIG. 18C indicating sample numbers of a sampling process carried out on the stethoscope transducer signal at a rate of 3000/second). As illustrated graphically in FIGS. 18A-18C, the sonographs 330, 340 and 350 are easily distinguishable and therefore useful to medical personnel, healthcare workers and caregivers as visual diagnostic tools.

In one embodiment, an electronic stethoscope system can include an alert system for generating an alert signal upon the occurrence of a specified condition. The alert system may be programmed into suitable software on a computer (such as computer 138 of FIG. 9) which receives the transducer signals from the electronic stethoscope head as discussed herein. For example, an alert system may include a detector function combined with a timing function, and may be programmed to generate an alert signal should the alert system fail to detect bowel sounds from the stethoscope head within a predetermined time interval. The software may be configured to allow a user to define the predetermined time interval, allowing a caretaker to select, for example, 15 seconds, 30 seconds, 45 seconds, or 60 seconds, etc. as the time interval after which, if no bowel sounds have been detected, an alarm signal is generated. The specific predetermined time interval is selected to suit the patient whose health is being monitored. The alert system may also include a default interval after which, if no bowel sounds have been detected, an alarm signal is generated. The alarm signal may be conveyed to caretakers as a call to check on the status of the patient.

In another embodiment, the alert system may generate an alert signal upon receiving bowel sounds which indicate a condition to which a caretaker's attention should be drawn. For example, the alert signal may be generated upon the detection of abnormal bowel sounds, e.g., sounds associated with GERD or necrotizing enterocolitis. For example, the computer detects the sonograph 350 or sonograph 340 as compared to the sonograph 330, recognized an unhealthy condition and triggers the alert signal to advise the caregiver to attend to the patient.

The electronic stethoscope systems described herein produce real-time analysis of bowel sounds for consideration as a vital sign in the diagnosis of certain patient conditions. In addition, the electronic stethoscope systems may optionally be configured to interface with, and/or may incorporate, other existing NICU equipment (“accessory devices”). Such other equipment can include, but is not limited to, electrocardiograms, respiration monitors and feeding systems. For example, in one embodiment, the electronic stethoscope system alert system may be configured to control an accessory device such as a feeding pump or a mechanized medication dispenser, and the alert system may be configured to generate a control signal to vary the operating status of the feeding pump or medication dispenser in response to identified patient conditions or bowel sounds. For example, the control signal may slow down, speed up, or stop the feeding pump or medication dispenser.

While the electronic stethoscope components and systems described herein have been shown for use with human medical patients, the invention is not limited in this regard, and in other embodiments these features may be used on animals, e.g., in veterinary settings, where they can be used to monitor the condition of an injured and/or dangerous animal. In addition, the features disclosed herein can be used to monitor mechanical devices from locations that are impossible or undesirable for human personnel to go. For example, a stethoscope head as described herein may be dropped or inserted into the rubble of a collapsed building or mine to help locate survivors by detecting and conditional signals received from the transducer.

The terms “first,” “second,” and the like, herein do not denote any order, quantity, or importance, but rather are used to distinguish one element from another. The terms “a” and “an” herein do not denote a limitation of quantity, but rather denote the presence of at least one of the referenced item.

Although the invention has been described with reference to particular embodiments thereof, it will be understood by one of ordinary skill in the art, upon a reading and understanding of the foregoing disclosure, that numerous variations and alterations to the disclosed embodiments will fall within the scope of this invention and of the appended claims.

What is claimed is:

1. An electronic stethoscope head comprising:
a head member having a contact surface for contact with a patient's body; and
a transducer in the head member; and
an adhesive on the contact surface.

2. The electronic stethoscope head of claim 1, wherein the head member comprises a bell.

3. The electronic stethoscope head of claim 1, wherein the head member comprises a diaphragm.

4. The electronic stethoscope head of claim 1, wherein the head member defines an air pressure equalization hole.

5. The electronic stethoscope head of claim 1, including a medical sensor on the head member.

6. The electronic stethoscope head of claim 1, comprising: a microphone on the head member positioned and configured to receive ambient sound.

7. A processing system for an electronic stethoscope, the processing system comprising:
an analog processing circuit configured to receive a transducer signal from a transducer and to be capable of amplifying and/or filtering the transducer signal, to yield a conditioned signal; and
a signal acquisition system configured to digitize the conditioned signal, to filter the digitized conditioned signal and to present the digitized conditioned signal to assist in patient diagnosis.

8. The processing system of claim 7, wherein the signal acquisition system operates best on a conditioned signal having a predetermined processing frequency band, and wherein the conditioning circuit is configured to remove components of the transducer signal which are outside of the predetermined processing frequency band.

9. The processing system of claim 8, wherein the analog processing circuit includes a low pass filter with an upper limit cutoff of about 1.6 kHz or less.

10. The processing system of claim 9, wherein the signal acquisition system comprises acquisition circuitry operably connected to the conditioning circuit.

11. The processing system of claim 10, wherein the signal acquisition system includes an external memory controller (EMC), a UART 156, input-output logic (IOL), a microprocessor and a block RAM (BRAM) memory.

12. The processing system of claim 10, wherein the signal acquisition system includes a compact Flash memory card and associated controller circuitry.

13. The processing system of claim 7, wherein the signal acquisition system is configured to apply frequency shifting and/or frequency stretching to the digitized conditioned signal to make inaudible portions of the digitized conditioned signal audible.

14. An electronic stethoscope system comprising:
an electronic stethoscope head comprising a head member having a contact surface for contact with a patient's body and a transducer; and
a processing system for an electronic stethoscope, the processing system comprising:
an analog processing circuit configured to receive a transducer signal from a transducer and to be capable of amplifying and/or filtering the transducer signal, to yield a conditioned signal; and
a signal acquisition system configured to digitize the conditioned signal, to filter the digitized conditioned signal and to present the digitized conditioned signal to assist in patient diagnosis.

15. The electronic stethoscope system of claim 14, wherein the signal acquisition system is configured to apply frequency shifting and/or frequency stretching to the digitized conditioned signal to make inaudible portions of the digitized conditioned signal audible.

16. The electronic stethoscope system of claim 14, including circuitry configured to produce an alarm signal when abnormal bowel sounds are detected or when no bowel sounds are detected for a predetermined interval.

17. The electronic stethoscope system of claim 14, including an accessory device and circuitry configured to produce a control signal to control the accessory device when abnormal bowel sounds are detected or when no bowel sounds are detected for a predetermined interval.

18. A method for detecting bodily sounds, comprising:
applying the stethoscope head of an electronic stethoscope system as described in claim 14 to the body of a patient;
generating a digitized conditioned signal from the stethoscope head, and
reconstructing an analog signal from the digitized conditioned signal.

19. A method for detecting bodily sounds, comprising:
applying the stethoscope head of an electronic stethoscope system as described in claim 14 to the body of a patient;
generating a digitized conditioned signal from the stethoscope head, and
applying frequency shifting and/or frequency stretching to the digitized conditioned signal.
